(19) United States
(12) Patent Application Publication
(10) Pub. No.: US 2022/0073859 A1
(43) Pub. Date: Mar. 10, 2022

(54) INTELLIGENCE-DRIVEN AUTOMATION OF BIOMASS EXTRACTION

(71) Applicant: Radient Technologies Innovations Inc., Edmonton (CA)

(72) Inventors: Steven Splinter, Edmonton (CA); Tomasz Popek, Edmonton (CA)

(21) Appl. No.: 17/238,855

(22) Filed: Apr. 23, 2021

Related U.S. Application Data
(63) Continuation of application No. PCT/IB2019/058877, filed on Oct. 17, 2019.
(60) Provisional application No. 62/749,547, filed on Oct. 23, 2018.
(60) Provisional application No. 62/749,561, filed on Oct. 23, 2018.

(51) Int. Cl. C12M 1/36; B01D 11/02; C12M 1/00; G05B 13/02

(52) U.S. Cl. CPC C12M 41/48; B01D 11/0207; B01D 11/0211; G05B 13/024; B01D 11/0288; B01D 11/0296; C12M 47/06; B01D 11/0226

(57) ABSTRACT

Methods and systems for intelligence-driven automation of biomass extraction are disclosed. A controller database may store controller data indicative of reference parameter values associated with functioning of processing chambers of the biomass extraction process, and such parameter values may be associated with obtaining extraction results from a biomass exhibiting certain properties. Real-time sensor data may be obtained from sensors connected to the processing chambers. The sensor data may be compared with the controller data to identify a requirement for altering the parameters. AI algorithms may be used to determine the optimal heating temperature, residence time of the biomass in the reactor, and other settings for other critical parameters of biomass extraction, including stages for biomass processing, decarboxylating, mixing with solvent(s), extraction (with microwave energy), filtering, formulation, etc.

### CROSS-REFERENCE TO RELATED APPLICATIONS

The present patent application is a continuation of International Application No. PCT/IB2019/058877 filed Oct. 17, 2019, which claims the priority benefit of U.S. provisional patent No. 62/749,547 filed Oct. 23, 2018 and U.S. provisional patent No. 62/749,561 filed Oct. 23, 2018, the disclosures of which are incorporated by reference herein.

### BACKGROUND OF THE INVENTION

### 1. Field of the Disclosure

The present disclosure is generally related to biomass extraction, and more particularly related to intelligence-driven automation of biomass extraction.

### 1. Description of the Related Art

The extraction of bioactive or therapeutic compounds from natural biomass sources, including for example plants or microoganisms, has been practiced throughout human history. Plant extracts may contain some or all of the benefits of the plant itself, in a convenient concentrated form. Such extracts are used for a wide variety of purposes, such as fuel, food, personal care and medicine. Conventional processes for biomass extraction typically include a plurality of processing chambers and units, such as a pre-processing preparation chambers, mixing chamber, extraction chamber, heating unit, filtration and separation chamber, solvent recovery chamber and several others. At industrial scale, such processes may require constant supervision from trained professionals to check upon several operating parameters related to the various stages of biomass extraction. Such supervision means that the overall biomass extraction process may not be scalable, efficient, or immediately responsive to real-time conditions. Thus, such a method that could automate a biomass extraction process is much desired.

One particular type of plant biomass—cannabis—is a genus belonging to the family of Cannabaceae, and has three main species Cannabis sativa, Cannabis Indica, and Cannabis ruderalis. The genus is indigenous to central Asia and the Indian subcontinent. Cannabis has a long history of being used for therapeutic and recreational purposes. The importance of cannabis in therapeutics is evidenced by the ever-increasing number of research publication related to the new indications for cannabis.

There is therefore a need for improved systems and methods of automating biomass extraction in a way that is driven by data and intelligence so as to optimize for desired parameters.

### SUMMARY OF THE CLAIMED INVENTION

Embodiments of the present invention provide systems and methods for intelligence-driven automation of biomass extraction In particular, parameters involved in the various stages of extraction may be determined, monitored, and adjusted as needed using historical data analysis, artificial intelligence and machine learning algorithms, and real-time sensor data collection. The result may provide an increase in efficiency, as well as more optimized extraction of cannabis (and/or other biomass).

In addition to improving efficiency of an extraction apparatus, such systems and methods may further enhance potency or purity of a final extract, maximize yield, and maximize extraction efficiency. Each biomass is natural product, and exhibits variances based upon the specific cultivar of the biomass, as well as the conditions in which the biomass was grown, harvested, shipped, and prepared for extraction. The raw incoming biomass can be analyzed and used as an input to determine the most efficient extraction parameters for that particular biomass sample. Over time, the algorithms for identifying parameters may be adjusted and refined as more data is collected about various raw biomass and respective extraction results.

### BRIEF DESCRIPTION OF THE DRAWINGS

FIG. 1 is a block diagram illustrating an exemplary system for intelligence-driven automation of biomass extraction.

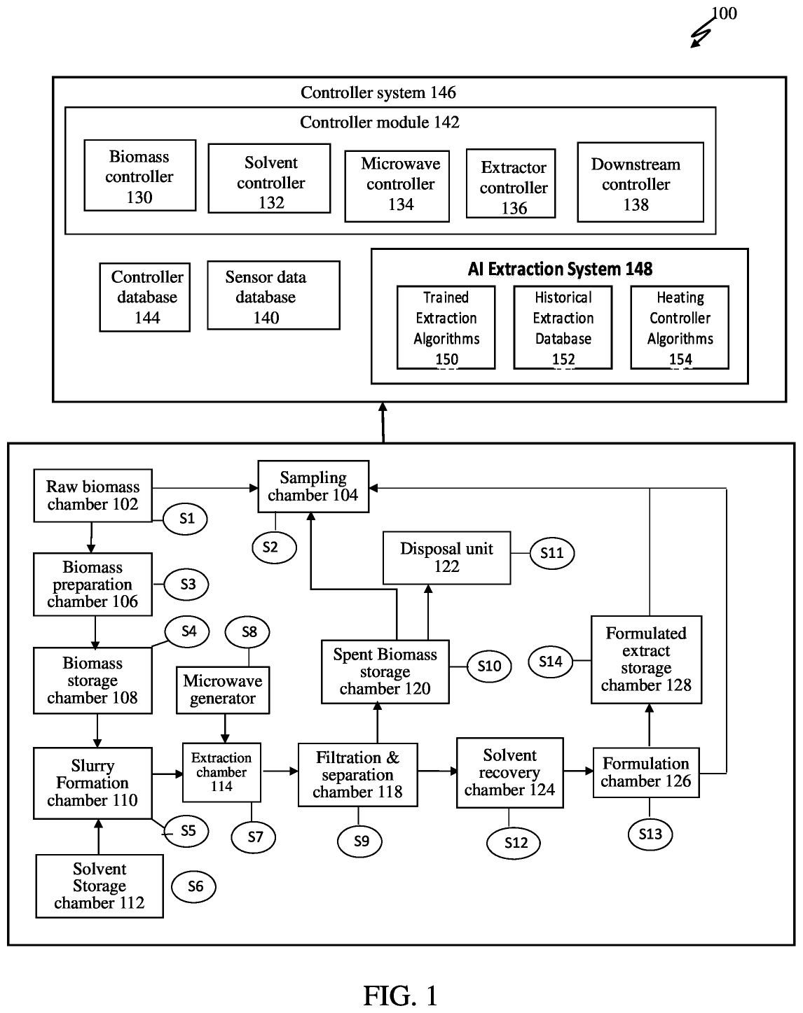

FIG. 2 is a flowchart illustrating an exemplary method of biomass extraction.

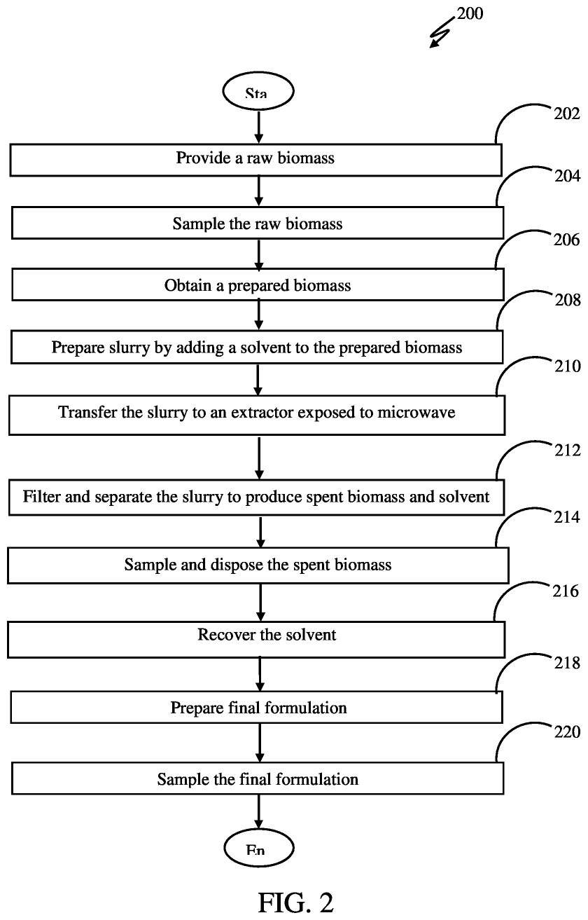

FIG. 3 is a flowchart illustrating an exemplary method for intelligence-driven automation of biomass extraction.

FIG. 4 is a flowchart illustrating an exemplary method for building a biomass profile database.

FIG. 5 is a flowchart illustrating an exemplary method for training algorithms for automated biomass extraction.

FIG. 6 illustrates an exemplary biomass profile database.

FIG. 7 is a flowchart illustrating an exemplary method for intelligence-driven automation of heating controls.

### DETAILED DESCRIPTION

Exemplary systems and methods for intelligence-drive automation of biomass extraction are provided. Information may be stored in memory regarding reference parameters for each stage of biomass processing (e.g., controlled conditions within the various processing chambers). By providing sensors in the various chambers involved in biomass processing, more precise calibrations and control may be achieved, thereby minimizing loss and maximizing throughput of desired components of a plant biomass.

The system for automating the biomass extraction process comprises a plurality of sensors that may be connected to processing chambers of a continuous flow processing apparatus. The processing chambers may include a raw biomass chamber 102, a sampling chamber104, a biomass preparation chamber 106, a biomass storage chamber 108, a slurry formation chamber 110, a solvent storage chamber 112, an extraction chamber 114, a microwave generator 116, a filtration and separation chamber 118, a spent biomass storage chamber 120, a disposal chamber 122, a solvent recovery chamber 124, a formulation chamber 126, and a formulated extract storage chamber 128. Conveyors between the various chambers provide a continuous flow of biomass—including biomass slurry—that is processed in different stages within the various different chambers. The rate of such flow and timing of each stage—as well as the stage-specific parameters within each chamber—may be intelligently controlled and adjusted in real-time based on real-time data, as well as correlations to historical biomass extraction data (e.g., conditions, results).

The plurality of sensors connected to the processing chambers may obtain sensor data related to parameters associated with the respective processing chamber. The parameters may include, but not limited to, a particle size, type of solvent, pressure of a solvent, flow rate of slurry, extraction temperature, extraction timing, microwave power, and conditions used (temperature, pressure, time spent) in recovering the solvent. It will be apparent to one skilled in the art that the above-mentioned parameters have been provided only for illustration purposes, without departing from the scope of the disclosure.

In step 202, a raw biomass may be provided and prepared in the raw biomass chamber102. The raw biomass may comprise various compounds targeted for extraction. The identified compounds may further affect what parameters are initially selected, as well as how such parameters may be adjusted. The raw biomass—which may include flowers (buds) form of cannabis plant—may be dried, grounded, or otherwise processed into particles of a specified size range. It should be noted that an average particle size of the raw biomass may range from 3-6 mm. Further, a sensor S1 may be connected to the raw biomass chamber 102 for collecting a real-time sensor data regarding properties of the raw biomass, which may be used as feedback to adjust operational parameters of the raw biomass chamber 102. In a case, the real-time sensor data may be related to a particle size of the raw biomass. Thereafter, the real-time sensor data may be monitored using a biomass controller 130.

Successively, the raw biomass may be sampled at step 204. The raw biomass may be sampled in the sampling chamber 104. In an embodiment, the raw biomass may be sampled and analyzed for determining cannabinoid content and a cannabinoid profile. The raw biomass may be sampled and analyzed using a suitable sampling and analytical technique, such as Ultra High performance Liquid Chromatography (UHPLC) technique. Further, various samples for gases may be analyzed using a Gas Chromatography-Mass Spectrometry Detection (GC-MS) technique. It should be noted that sampling of the raw biomass may reveal a sample profile of the raw biomass. Further, a sensor S2 may be connected to the sampling chamber 104 for collecting real-time sensor data used to control and adjust parameters associated with the sampling chamber 104. Thereafter, the real-time sensor data may be monitored using the biomass controller 130.

Successively, the raw biomass may be subjected to a preparation step, such as decarboxylation by heating in biomass preparation chamber 106, thereby resulting in a prepared biomass at step 206 . . . The prepared biomass may be stored in the biomass storage chamber 108 until a controlled continuous flow may be transported to the next processing chamber. Further, sensor S3 and S4 may be connected to the biomass preparation chamber 106 and the biomass storage chamber 108, respectively. Such sensors may collect real-time sensor data related to various properties exhibited by the prepared biomass. Thereafter, the real-time sensor data may be monitored using the biomass controller 130.

After preparing (e.g., decarboxylating) the biomass, the prepared biomass may be used to form a slurry in step 208. The slurry may be prepared in the slurry formation chamber 110 by adding a solvent to the prepared biomass. The solvent added to the prepared biomass may be selected based on different dielectric and solvent parameter properties. The solvent may be selected from an alcohol group (e.g., ethanol, isopropanol), alkane group (e.g., pentane), and ketone group (e.g., acetone, butanone), stored in the solvent storage chamber 112. The solvent-to-biomass ratio may be maintained at 10 1/kg to ease pumping operation of the slurry. Further, a sensor S5 may be connected to the slurry formation chamber 110 for collecting real-time sensor data for controlling parameters associated with the slurry formation chamber 110. In some embodiments, a sensor S6 connected to the solvent storage chamber 112 may collect real-time sensor data used to identify optimal parameters associated with the solvent storage chamber 112. For example, type, composition, temperature, pressure, etc., of the solvent may be recorded by the sensor S6. Thereafter, the real-time sensor data may be monitored using a solvent controller 132.

Successively, the slurry may be transferred to the extraction chamber 114 at step 210. The slurry may be transported into the extraction chamber 114 in a continuous mass flow, which may be provided via a set of mechanical conveyors, worm gear, or other controlled flow transport. Further, a sensor S7 may be connected to the extraction chamber 114 for collecting be provided via a set of mechanical conveyors, worm gear, or other controlled flow transport. Further, a sensor S7 may be connected to the extraction chamber 114 for collecting real-time sensor data for controlling parameters associated with the extraction chamber 114. In an example, a rate of flow of the slurry may be captured by the sensor S7. Thereafter, the real-time sensor data may be monitored using an extraction chamber controller 136. Further, the slurry may be subjected to a thermal processing (e.g., microwave heating by a microwave generator116). Further, a sensor S8 may be connected to the microwave generator 116 for collecting real-time sensor data for controlling parameters associated with the microwave generator116. In an example, microwave power may be captured by the sensor S8. Thereafter, the real-time sensor data may be monitored using a microwave controller 134. In one case, the slurry may be transported into a reactor portion (e.g., tube) within the extraction chamber 114. At least one portion of the extraction chamber 114 or the entire chamber may be the microwave-transparent, thereby allowing the flow of slurry to be exposed to the microwave energy generated by microwave generator 116. Conveyors within the extraction chamber 114 may allow for a continuous flow of the slurry to be exposed to the microwave energy under controlled conditions (e.g., microwave power, temperatures, times, etc.), which may be further adjusted based on observed properties of the processed biomass or slurry.

When the continuous flow of slurry emerges from the extraction chamber (after exposure to microwave), various targeted components will have been extracted from the biomass into the solvent within the slurry. As such, the slurry flowing out of the extraction chamber 114 may be comprised of the biomass—now spent—and a solvent-extract mixture.

The spent biomass and solvent-extract mixture may be filtered and separated in step 212. Such filtration may be performed by the filtration and separation chamber 118. Thereafter, the spent biomass may be stored in the spent biomass storage chamber 120. It should be noted that the separation may be performed using filtration, centrifugation, and other similar processes. A sensor S9 may be connected to the filtration and separation chamber 118 for collecting real-time sensor data regarding properties of the unfiltered slurry, as well as the separated components following filtration. Such properties may be used to adjust and refine the parameters of the filtration and separation chamber 118 for subsequent parts of the flow. Further, a sensor S10 may be connected to the spent biomass storage chamber 120 for collecting real-time sensor data for controlling parameters associated with the spent biomass storage chamber 120. Thereafter, the real-time sensor data may be monitored using a downstream controller 138.

Successively, the spent biomass may be sampled and analyzed in step 214. Sampling and analysis of the spent biomass may be performed by the sampling chamber 104. The spent biomass may be analyzed using several analtyical techniques. In an embodiment, analysis of cannabinoid content and cannabinoid profile may be performed using an Ultra High Performance Liquid Chromatography (UHPLC). Further, analysis of various samples of terpene profile may be performed using a Gas Chromatography-Mass Spectrometry Detection (GC-MS) technique. Further, the sensor S2 connected to the sampling chamber 104 may be used for collecting real-time sensor data related to the sampling data 104. Thereafter, the real-time sensor data may be monitored using the downstream controller 138.

After sampling and analyzing the spent biomass, disposed composition or waste spent biomass may be incinerated or mixed with a deactivating agent for disposal in the disposal chamber 122. In one case, clay may be used as the deactivating agent. Further, a sensor S11 may be connected to the disposal chamber 122 for collecting a real-time sensor data. Thereafter, the real-time sensor data may be monitored using the downstream controller 138.

The solvent may be recovered from the solvent-extract mixture by the solvent recovery chamber 124. In an embodiment, the solvent may be recovered using a distillation process. Upon recovery, the solvent may be used in another extraction process. Further, a sensor S12 may be connected to the solvent recovery chamber 124 for collecting a real-time sensor data regarding properties of the solvent and the conditions used for solvent recovery (e.g. temperature, pressure, time); such sensor data may be used to adjust parameters associated with the solvent recovery chamber 124. Thereafter, the real-time sensor data may be monitored using the downstream controller 138.

Successively, a final formulation (e.g., a final formulated extract) may be prepared at step 218. The final formulation may be prepared in the formulation chamber 126. It should be noted that the de-solventized extract may be formulated into a final formulated extract using a suitable formulation method. The final formulated extract may be stored in a formulated extract storage chamber 128. Thereafter, the final formulated extract may be sampled and analyzed, at step 220. The sampling and analysis may be done using the sampling chamber 104. The final formulated extract may be sampled and analyzed using a suitable technique, such as an Ultra High Performance Liquid Chromatography(UHPLC). Various samples of the terpene profile may be detected and analyzed using a Gas Chromatography-Mass Spectrometry Detection (GC-MS). The sampling techniques may help to determine the cannabinoid content and the cannabinoid profile of the final formulated extract. Further, sensors S13 and S14 may be connected to the formulation chamber 126 and the formulated extract storage chamber 128 respectively; such sensors may collect real-time sensor data used to control parameters associated with the formulation chamber 126 and the formulated extract storage chamber 128, respectively. Thereafter, the real-time sensor data may be monitored using the downstream controller 138.

It should be noted that the sensor data for the respective properties of the biomass, slurry, and extract in various stages may be used to correlate to properties of historical flows in conjunction with parameters resulting in such properties. Such data may be used to identify the parameters for controlling conditions in each processing chamber (102 to 128) and stored in a sensor data database 140 for future reference. The sensor data stored in the sensor data database 140 is mentioned in below provided Table 1:

TABLE 1
Sensor data stored in sensor data database

| Sensor | Parameter | Controller | Real-time input | |
|---|---|---|---|---|
| S3 | Particle size | Biomass Controller | 2.5 | mm |
| S5 | Solvent temperature | Solvent Controller | 15° | C. |
| S7 | Rate of flow for slurry | Extraction chamber | 2 | L/minute |
| | | Controller | | |
| S6 | Power | Microwave | Medium | |
| | | Controller | | |
| . . . | . . . | . . . | . . . | |
| S9 | Time spent in solvent | Downstream | 45 | minutes |
| | recovery | Controller | | |

Successively, a controller module 142 may retrieve the sensor data from the sensor data database 140. Successively, the controller module 142 may compare the sensor data with controller data to identify a requirement for altering the parameters. The controller data may be indicative of reference parameter values associated with functioning of the processing chambers of the biomass extraction process. The controller data may be stored in a controller database 144. The controller data stored in the controller database 144 is mentioned in below provided Table 2:

TABLE 2
Controller data stored in controller database

| Parameter | Controller | Standard Parameter |
|---|---|---|
| Particle Size | Biomass | >6 mm, large particle |
| | Controller | 3-6 mm, standard |
| | | <3 mm, small particle |
| Solvent Temperature | Solvent | <20° C., low temperature |
| | Controller | 20-22° C., standard |
| | | >22° C., high temperature |
| Flow rate of slurry | Extraction | <5 l/minute, slow |
| | chamber | 5 L/minute standard |
| | Controller | >5 L/minute, fast |
| Temperature | Microwave | <30° C., low temperature |
| | Controller | 30° C.-32° C., standard |
| | | >32° C., high temperature |
| . . . | . . . | . . . |
| Time spent in | Downstream | <60 minutes, less than standard |
| solvent recovery | Controller | 60-70 minutes, standard |
| | | >70 minutes, more than standard |

Thereafter, based on the requirement, the controller module 142 may regulate at least one processing chamber of the processing chambers by sending a feedback. The controller module 142 may regulate various controllers, such as the biomass controller 130, the solvent controller 132, the microwave controller 134, the extraction chamber controller 136, and the downstream controller 138 for setting and adjusting the parameters associated with each of the processing chambers 102 to 128. In an example, different cultivars may be associated with certain compounds having properties that were achieved under a recorded set of parameters. Different properties at each stage and associated parameters may therefore be associated with certain results, such as efficiency of extraction, purity, potency of the extract, organoleptic properties of the extract, and other quantitative and qualitative properties. Such associations and correlations may therefore be used to set operational parameters of each processing chamber, as well as individually adjust throughout based on observed properties of the flow currently being processed within the respective chamber.

For example, sensor data may be collected to identify a particle size range within a flow portion by sensor S3. Based on such sensor data, the controller module 142 may activate the biomass controller 130 to adjust the parameters associated with the biomass controller 130. Similarly, sensor data gathered from each processing chamber may be used to control operational parameters not only of the processing chamber associated with the sensor, but also the parameters of upstream and downstream chambers,

Controller system 146 may include controller module 142 (inclusive of biomass controller 130, but also solvent controller 132, microwave controller 134, extractor controller 136, downstream controller, 138), the controller database 144, the sensor database 146, and an artificial intelligent (AI) extraction system 148 (including trained extraction algorithms 150, historical extraction database 152, and heating controller algorithms 154).

Trained extraction algorithms of the AI extraction system 148 may be executable to identify recommended parameters for the various stages of extraction process based on analysis of the observed properties of the incoming biomass. Such trained algorithms may identify correlations in historical data so as to identify the parameters applied to past biomass/slurry with similar properties that have resulted in a certain result (e.g., high retention of organoleptic properties).

A historical extraction database 152 of the AI extraction system 148 may contain data of previous extractions, including analysis of the biomass, parameters of the extraction, and analysis of the extract, etc., that the trained extraction algorithms can draw from to determine extraction parameters for a subsequent biomass/slurry flow at step 136. The sensor data being gather in real-time may also be added to the historical extraction database 152, as well as the specified parameters and associated sampling data regarding the results thereof. As such, historical database 152 may be continually updated so as to provide as granular results as possible in matching or correlating a current biomass to past iterations of biomass processed under different conditions, as well as identifying which conditions (e.g. parameters) resulted in more desirable outcomes.

A heating controller algorithm may specifically be used to analyze real-time sensor data, correlate to past biomass, and select for parameters correlated to a desired result. The selected parameters may thereafter be sued to control the heating chamber. The heating controller chamber allows for precise control over the extraction chamber 114 (e.g., so that cannabinoid compounds may be extracted while avoiding co-extraction of unwanted compounds). An analysis of a sample biomass that is used by the trained extraction algorithm 150 to determine extraction parameters in order to create a desired extract. The extraction parameters determined by the trained extraction algorithms 150 by computing the analysis of the sample of biomass using an algorithm that has been trained on historical extraction data from the historical extraction database 152.

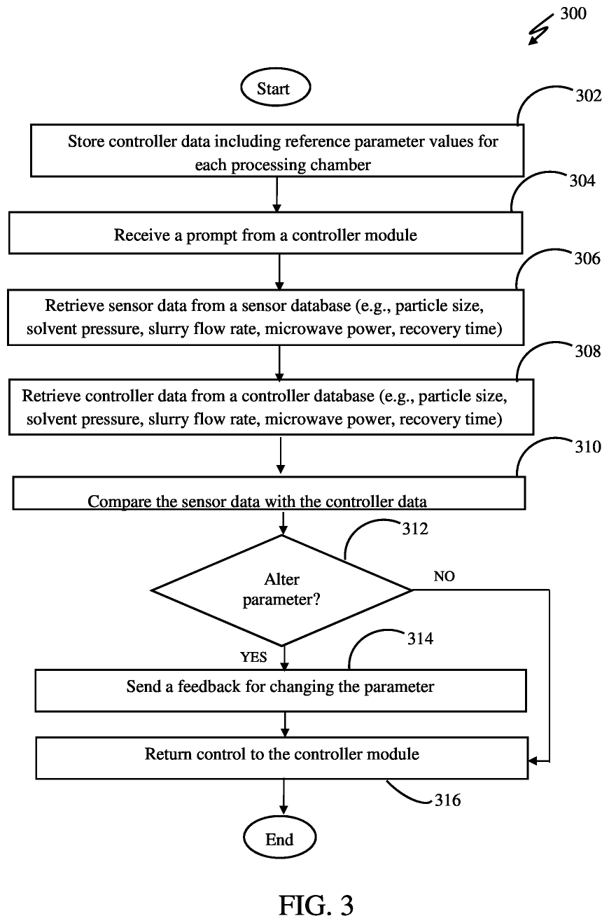

FIG. 3 illustrates a flowchart 300 illustrating a method performed by controller system 146. Functioning of the controller system 146 will now be explained with reference to flowchart 300 shown in FIG. 3. One skilled in the art will appreciate that, for this and other processes and methods disclosed herein, the functions performed in the processes and methods may be implemented in differing order. Furthermore, the outlined steps and operations are only provided as examples, and some of the and operations may be optional, combined into fewer steps and operations, or expanded into additional steps and operations without detracting from the essence of the disclosed embodiments.

Controller data may include reference parameter values for each processing chamber and may be stored in controller database 144. Such reference parameters may further be associated with the data regarding past biomasses, various properties thereof, and extraction results. In step 304, controller module 142 may prompt for retrieval of sensor data from the various sensors located at different locations (e.g., different processing chambers) to monitor the different stages of the continuation biomass extraction flow. Successively, the controller module 142 (or specific controllers thereof, including biomass controller 130, solvent controller 132, microwave controller 134, extractor controller 136, or downstream controller 138) may retrieve sensor data from the sensors themselves or from sensor data database 140.

The sensor data may correspond to a particle size (e.g., a real-time particle size) of the prepared biomass, solvent pressure, slurry flow rate, microwave power, recovery time, or other property of the components of the biomass extraction system. Successively, the controller module 142 may retrieve controller data from the controller database 144 at step 306. The controller data may include operational parameters of a processing chamber in order to achieve a type of result. For example, a given particle size of the prepared biomass may be associated with a desired level of efficiency in extracting one or more target components from a given cultivar. Other particle sizes may be associated with desired efficiency levels for other components or other cultivars. Likewise, other types of operational parameters may be associated with achieving desired efficiency rates in view of the specific solvent, slurry, biomass, etc., combination that exhibits certain properties.

Successively, the controller module 142 may compare the sensor data with the controller data at step 308. Based on the comparison, the controller module 142 may identify a need to alter any parameter of any of the processing chambers. For example, current operational parameters may be identified as having been associated with subpar extraction efficiency, purity, potency, etc., in past biomass flows. Where a current set of properties and parameters is identified as having such room for improvement, the parameters may be adjusted in accordance with historical data regarding parameter values associated with better results.

As data is collected and stored in controller data database 144, sensor data database 140, and historical extraction database 152, such data may be analyzed in conjunction to identify trends and patterns among the different properties detected by the sensors and/or provided via sampling. Such trends and patterns may be subject to trained extraction algorithms 150 and heating controller algorithms 154 to predict what operational parameters are most likely to result in a desired outcome (e.g., high extraction efficiency levels, purity, potency of specific target extract, specific organoleptic property, a qualitative property such as pleasing taste, smell, etc.).

Based on the checking, the controller module 142 may send a feedback for changing the parameter to the associated chamber(s) at step 312. It should be noted that changes in the parameter may be sent through a relevant control line. Thereafter, the controller module 142 may return to retrieving and analyzing subsequent sensor data in order to identify whether further adjustment may be needed.

In an example, a real-time particle size of the prepared biomass may be 2.5 mm, stored in the sensor data database 140. On the other hand, a standard particle size of the prepared biomass may be set as a range from 3-6 mm, stored in the controller database 144. The biomass controller 130 may retrieve the real-time particle size and the standard particle size from the sensor data database 140 and the controller database 144 respectively. Successively, the biomass controller 130 may perform a comparison between values of the real-time particle size and the standard particle size. Based on the comparison, the biomass controller 130 may determine that a value of the real-time particle size lies outside a range of the standard particle size. Thereafter, based on such determination, the biomass controller 130 may change parameters, such as microwave power or the flow rate of the slurry through the extraction chamber 114.

Similarly, a real-time temperature of the solvent may be 15° C. and stored in the sensor data database 140. On the other hand, a standard temperature of the solvent may be set as a range from 20-22° C., stored in the controller database 144. The solvent controller 132 may retrieve the real-time temperature of the solvent and the standard temperature of the solvent from the sensor data database 140 and the controller database 144 respectively. Successively, the solvent controller 132 may perform a comparison between values of the real-time temperature of the solvent and the standard temperature of the solvent. Based on the comparison, the solvent controller 132 may determine that a value of the real-time temperature of the solvent lies outside the range of the standard temperature of the solvent. Thereafter, based on the determination, the solvent controller 132 may change the parameter such as microwave power.

In another example, a real-time flow rate of the slurry may be 2 L/minute, stored in the sensor data database 140. On the other hand, a reference flow rate of the slurry may be 5 L/minute, stored in the controller database 144. The extraction chamber controller 136 may retrieve the real-time flow rate of the slurry and the standard flow rate set for the slurry from the sensor data database 140 and the controller database 144 respectively. Successively, the extraction chamber controller 136 may perform a comparison between values of the real-time flow rate of the slurry and the standard flow rate of the slurry. Based on the comparison, the extraction chamber controller 136 may determine that a value of the real-time flow rate of the slurry is less than the standard flow rate of the slurry. Thereafter, based on the determination, the extraction chamber controller 136 may change the parameter such as microwave power (e.g., reducing the microwave power to compensate for reduced real-time flow rate of the slurry).

Further, real-time power of the microwave may be identified by sensors and stored in the sensor data database 140. On the other hand, a standard power of the microwave may generally be set and stored in the controller database 144. The microwave controller 134 may retrieve the real-time power of the microwave and the standard power of the microwave from the sensor data database 140 and the controller database 144 respectively. Successively, the microwave controller 134 may perform a comparison between values of the real-time power of the microwave and the standard power of the microwave. Based on the comparison, the microwave controller 134 may determine that a value of the real-time power of the microwave lies outside a range of the standard power of the microwave. Thereafter, based on the determination, the microwave controller 134 may increase a flow rate of the extraction chamber 114, and thus ensure that the slurry is heated to a proper temperature.

In an example, a time spent in recovering a solvent in a real-time may be 45 minutes, stored in the sensor data database 140. On the other hand, a standard time spent in recovering a solvent may be set at 60-70 minutes and stored in the controller database 144. The downstream controller 138 may retrieve the time spent in recovering the solvent in a real-time and the standard time spent in recovering the solvent from the sensor data database 140 and the controller database 144 respectively. Successively, the downstream controller 138 may perform a comparison between the time spent in recovering the solvent in a real-time and the standard time spent in recovering the solvent. Based on the comparison, the downstream controller 138 may determine that a value of the time spent in recovering the solvent in a real-time lies outside a range of the standard time spent in recovering the solvent. Thereafter, based on the determination, the downstream controller 138 may modify a method for formulating the final extract in such a way that more solvent may be present in the extract.

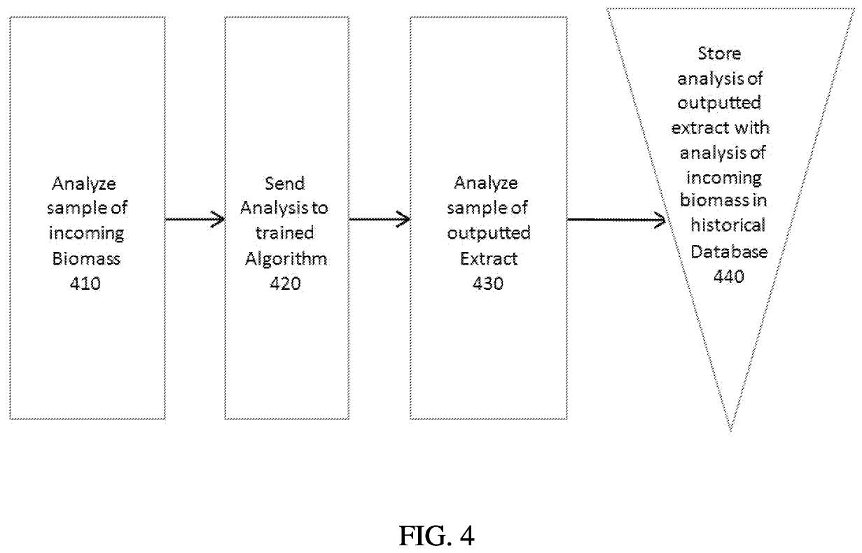

FIG. 4 is a flowchart illustrating an exemplary method 400 for building a biomass profile database. Such biomass profile database may result from successive testing by sampling chamber 104.

In step 410, a sample of incoming biomass (e.g., cannabis) may be analyzed. Send Analysis to trained algorithm, which may be, for example, an analysis of the concentration and ratio of various cannabinoids (e.g., THC, CBD, CBG, CBN, etc.), terpenes, flavonoids, moisture concentration, or other features of the cannabis biomass at step 420. A sample of outputted extract may be analyzed, which may be, for example, an analysis of the concentration and ratio of various cannabinoids of the extracts, terpenes, flavonoids, viscosity, color, etc. at step 430, and the analysis of outputted extract with analysis of incoming biomass may be stored in historical database 152—which may further include comparisons of the biomass and outputted extract, how close was the cannabinoid profile of the biomass to the outputted extract, etc.—at step 440.

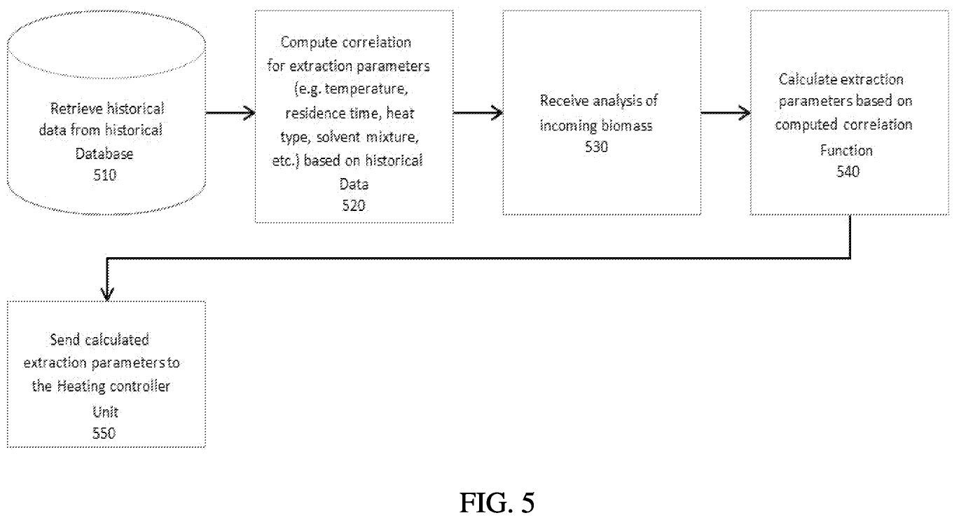

FIG. 5 is a flowchart illustrating an exemplary method 500 for training algorithms for automated biomass extraction. In step 510, historical data may be retrieved from historical database 152 (e.g., data collected from all previous extraction runs, including sampling data, sensor data, and extraction parameter data).

In step 520, correlations for extraction parameters (e.g. temperature, residence time, solvent composition, etc.) may be identified based on historical data. In some embodiments the correlation is computed using one of several statistical methods (e.g., ordinary least squares, logistic regression, Pearson's product-moment correlation, Spearman's correlation coefficient rho, etc.).

In step 530, analysis of incoming biomass may be received , which may include an analysis of the concentration and ratio of various cannabinoids (e.g., THC, CBD, CBG, CBN, etc.), terpenes, flavonoids, moisture concentration, or other features of the cannabis biomass). In step 540, extraction parameters (e.g., preheat, extraction, and cool down parameters for the heating controller) may be calculated based on computed correlation function). Such calculation may be based on inputting the biomass analysis as independent variables into a function generated by the correlation computed identified in step 520. Such calculated extraction parameters may be sent to the heating controller 154in step 550.

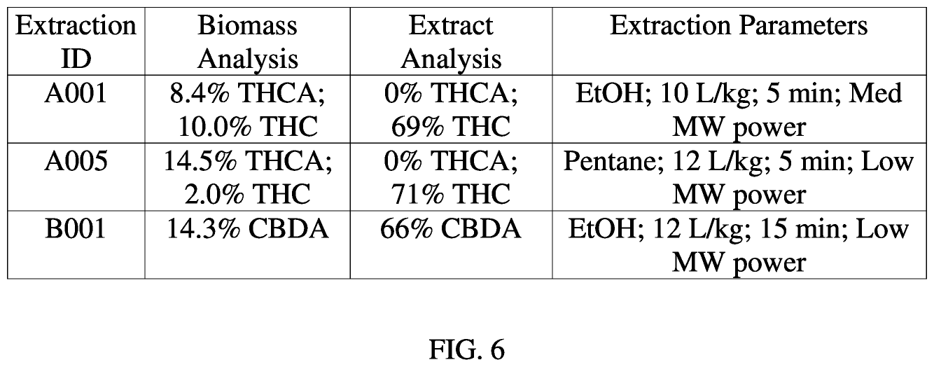

FIG. 6 illustrates entries that may be included in an exemplary biomass profile database for storing historical data. The historical database is a database that includes the analysis of extracted biomass, the parameters used to extract the biomass, and an analysis of the final extract. The historical database may be used by the AI extraction system 148 to train an algorithm. Over time, additional entries may be added to the historical database, and the type of data from such entries may further include additional characteristics of the respective biomass and/or extract.

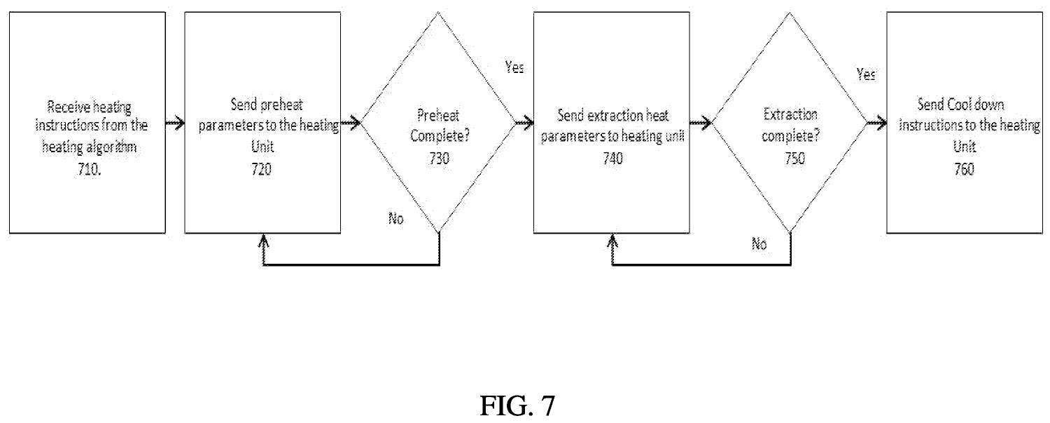

FIG. 7 is a flowchart illustrating an exemplary method 700 for intelligence-driven automation of heating controls. In step 710, heating instructions may be received from the trained extraction algorithms 150, which may include parameters used to preheat, set extraction conditions, and cool down.

In step 720, preheat parameters may be sent to a heating chamber and initiating the heating sequence. In some embodiments, the heating sequence may instead be a cooling sequence. The heating controller may be tested to see if the preheating operation is complete at step 730. If yes, then the heating controller chamber begins a step where the heating controller chamber sends the extraction heat parameters to the heating chamber, thereby beginning the extraction phase of the heating sequence in step 740.

The heating controller may also test to see if the extraction operation is complete at step 750. If yes, then the heating controller chamber begins a step where the heating controller chamber sends cool down instructions to the heating chamber, completing the heating sequence, at step 760.

Moreover, although the present disclosure and its advantages have been described in detail, it should be understood that various changes, substitutions and alterations can be made herein without departing from the disclosure as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method for automating a biomass extraction process, the method comprising:
storing data in memory regarding a plurality of parameter values associated with a set of desired properties;
receiving sensor data captured by a plurality of sensors each located at one of a plurality of different processing chambers of a continuous flow extraction apparatus;
analyzing the sensor data to identify one or more properties observed within the respective processing chamber;
predicting, based on the identified properties, that one or more of the stored parameter values are associated with producing the desired properties; and
regulating at least one of the processing chambers based on the prediction, wherein the operations of the at least one processing chamber are adjusted in accordance with the one or more predicted parameter values associated with the desired properties.

2. The method of claim 1, wherein the identified properties include at least one of particle size, temperature of a solvent, flow rate of slurry, temperature, microwave power, and time spent in recovering the solvent.

3. The method of claim 1, further comprising storing the received sensor data in a database in memory.

4. The method of claim 3, further comprising sampling a resulting extract to obtain extract data, and storing the extract data in association with the sensor data and the one or more parameter values.

5. The method of claim 1, wherein the sensor data is received in real-time from one or more of the sensors located at one of the processing chambers, and wherein regulating the at least one processing chamber includes regulating an upstream chamber or a downstream chamber from the at least one processing chamber.

6. The method of claim 1, wherein regulating the at least one processing chamber includes modifying at least one current parameter of the at least one processing chamber.

7. The method of claim 1, further comprising setting the parameter values stored in memory as a standard, and updating the standard based on subsequent sampling.

8. The method of claim 1, wherein the prediction further includes an extraction result based on the identified properties, wherein the prediction indicates a quality level below a desired threshold level.

9. The method of claim 8, wherein regulating the at least one processing chamber is based on the predicted quality level.

10. An apparatus for automating a biomass extraction process, the apparatus comprising:
memory that stores data regarding a plurality of parameter values associated with a set of desired properties; and
a controller that:
receives sensor data captured by a plurality of sensors each located at one of a plurality of different processing chambers of a continuous flow extraction apparatus;
analyzes the sensor data to identify one or more properties observed within the respective processing chamber,
predicting, based on the identified properties, that one or more of the parameter values are associated with producing the desired properties; and
regulates at least one of the processing chambers based on the prediction, wherein the operations of the at least one processing chamber is adjusted in accordance with the identified one or more parameter values associated with the desired properties.

11. The system of claim 10, wherein the identified properties include ast least one of particle size, temperature of a solvent, flow rate of slurry, temperature, microwave power, and time spent in recovering the solvent.

12. The system of claim 10, wherein the memory further stores the received sensor data.

13. The system of claim 12, further comprising a sampling chamber that samples a resulting extract to obtain extract data, and wherein the memory further stores the extract data in association with the sensor data and the one or more parameter values.

14. The system of claim 10, wherein the sensor data is received in real-time from one or more of the sensors located at one of the processing chambers, and wherein the controller regulates the at least one processing chamber by regulating an upstream chamber or a downstream chamber from the at least one processing chamber.

15. The system of claim 10, wherein the controller regulates the at least one processing chamber by modifying at least one current parameter of the at least one processing chamber.

16. The system of claim 10, wherein the controller further sets the parameter values stored in memory as a standard, and updates the standard based on subsequent sampling.

17. The system of claim 10, wherein the prediction further concerns an extraction result based on the identified properties, wherein the prediction indicates a quality level below a desired threshold level.

18. The system of claim 17, wherein the controller regulates the at least one processing chamber is based on the predicted quality level.
